(19) United States
(12) Patent Application Publication
(10) Pub. No.: US 2013/0272923 A1
(43) Pub. Date: Oct. 17, 2013

(54) LIQUID DISPENSER WITH OZONATING, RECIRCULATING AND IMPROVED TEMPERATURE CONTROL FUNCTIONS

(71) Applicants: Chun Yen Wang, El Paso, TX (US); Gregory N. Spear, Los Angeles, CA (US)

(72) Inventors: Chun Yen Wang, El Paso, TX (US); Gregory N. Spear, Los Angeles, CA (US)

(73) Assignee: MTN PRODUCTS, INC., LaVerne, CA (US)

(21) Appl. No.: 13/445,030

(22) Filed: Apr. 12, 2012

(51) Int. Cl. A61L 2/20

(52) U.S. Cl. CPC A61L 2/202
USPC 422/29; 222/148

(57) ABSTRACT

Liquid dispensing systems which sanitize the liquid and liquid-contacting parts of a liquid dispensing system using ozonation, by periodically flushing the system with ozonated gas or liquid. Also disclosed and claimed are an apparatus and method for controlling temperature variations between the liquid in holding (e.g., cold and/or hot) tanks and the liquid as it is dispensed, which may be used with an Insta-Boil feature.

### INCORPORATION BY REFERENCE

The following copending patents and patent applications are incorporated by reference in their entirety into this patent application: co-pending U.S. Ser. No. ______, titled “Liquid Dispenser With Sanitizing Control Functions,” filed on the same date as the instant application; U.S. Pat. No. 7,434,603 (“BL 1”); U.S. Ser. No. 12/056,038, filed Mar. 26, 2008 and titled “Bottom Load Water Cooler” (“BL 2”); U.S. Ser. No. 12/116,407, filed May 7, 2008 and titled “Bottom Load Water Cooler” (“BL 3”); U.S. Ser. No. 12/555/866, filed Sep. 9, 2009 and titled “Energy Saving Baffle For Water Cooler”; and U.S. Ser. No. 12/611,221, filed Nov. 3, 2009 and titled “No-Spill Liquid Dispenser.”

The following patents are incorporated by reference herein with regard to their disclosure concerning ozonation processes for liquid dispensing systems: U.S. Pat. Nos. 6,561,382; 7,114,637; 7,422,684; 7,748,233 and 7,640,766.

### BACKGROUND OF THE INVENTION

The present invention generally relates to water coolers drawing water from either a bottle, or from point-of-use (POU) filtered water. More specifically, the invention relates to systems and apparatus with sanitizing functions (e.g., hot water flush and/or ozone use) and improved temperature control functions (e.g., providing enhanced control over first draw temperature, etc.).

Two main dispensing methods are used to dispense water or another liquid from a water cooler to a vessel such as a cup or glass: gravity-fed and pump-fed methods. With gravity-fed methods, a dispensing nozzle is positioned sufficiently lower than the water level of the water tank to allow water to be dispensed to the vessel at a desired flow rate based on gravity. With pump-fed systems, the dispensing nozzle may be positioned higher than the water level of the tank, and a pump or similar apparatus may be used to cause the liquid to flow to the nozzle area at a desired flow rate for dispensing to the vessel. In addition to a pump, electronics and valves are usually needed with pump-fed systems.

Gravity-fed systems typically position a faucet or other dispensing nozzle close to the water tank, so there is usually less variation in temperature between the liquid in the tank and the liquid being dispensed from the faucet, than in pump-fed systems. However, in both types of systems, this can be an issue. In addition, experience in the water cooler and liquid dispensing industry has shown that water cooler assemblies and liquid dispensing apparatus with sanitizing functions and/or improved temperature control functions would be advantageous, both for pathogen control and also to better control “first draw” temperatures and thus increase customer satisfaction.

### Definition of Claim Terms

The following terms are used in the claims of the patent as filed and are intended to have their broadest meaning consistent with the requirements of law. Where alternative meanings are possible, the broadest meaning is intended. All words used in the claims are intended to be used in the normal, customary usage of grammar and the English language.

“Sanitize” means to heat a liquid or gas to a sufficient temperature, or to apply a pathogen-killing material such as ozone, sufficient to kill pathogens in a liquid to be dispensed, to ensure the safety of liquid for ingestion by humans. As to heated water or steam, “sanitize” means heating the water to at least between 165° F.-180° F., for a sufficient time period to accomplish this result, and consistent with United States FDA specifications (Food Establishment Plan Review Guide—Section III, Part 9).

“Waterways” means any pathways (such as but not limited to tubing or “liquid communication means”) through which liquid in a liquid dispensing system runs, including cold and hot waterways, pumps, solenoid valves and other wetted parts. (“Liquid” as used here is not limited to “water,” and may include fruit juices and other non-alcoholic as well as alcoholic beverages.) As an example, in the preferred embodiment described below, all the waterways may be sanitized by ozonated water in the “Full Clean” mode described below, as the ozonated water will be circulated through the normal dispensing cold water pathways, and also through the faucet/nozzle/dispensing mechanism. As a further example, in the “Auto Clean Mode” described below, there may be a small section (from the Accu-Temp™ valve to the nozzle, for example) which may not be sanitized.

### SUMMARY OF THE INVENTION

The objects mentioned above, as well as other objects, are solved by the present invention, which overcomes disadvantages of prior water cooler assemblies and liquid dispensing apparatus, while providing new advantages not believed associated with such assemblies and apparatus.

In one group of preferred embodiments, a liquid dispensing system is disclosed in which ozonated water is moved through the water ways of the system to sanitize all or some subset of all of the wetted, liquid-contacting parts of the system.

### Accu-Temp™

Liquid dispensing systems such as water coolers are typically designed to meet certain temperature specifications (e.g., about 45° F. for cold water and about 185° F. for hot water). If water has been in the waterways for an extended period of time because no customers have drawn water recently, a common problem with liquid dispensing systems is that the next customer dispensing liquid may receive an initial liquid draw (known in the industry as the “first draw” temperature) that is not at the proper temperature specification. With liquid dispensing systems of the present invention, water may be periodically circulated through the waterways of the dispensing system to ensure that the water or other liquid to be dispensed in the waterways does not become contaminated and that the “first draw” temperature is within temperature specifications.

The demand to have higher dispensing heights, faster flow rates (faster fill time) and a more aesthetic appealing design for a water cooler or liquid dispensing system has made it difficult for conventional “gravity fed” systems to compete with “pump-fed” water cooler systems. However, because pump-fed systems require longer tubing pathways to connect between the nozzle and the water tanks, these systems are commonly associated with complaints about the first draw water temperature (e.g., too warm for cold water, too tepid for hot water). One problem associated with the first draw temperature not being to specification stems from the fact that the new water from the water tank may be mixed with the residual room temperature water residing in the water tubing since previous dispensing. Given typical ambient temperatures where water cooler systems are located, cold water sitting in the tubing will grow warmer, and hot water will grow cooler, eventually reaching room temperature (or wanner), even with good insulation. Shortening the tubing length will improve first draw temperatures, but a considerable length of tubing is still required for a pump-fed system to work properly.

Accordingly, the present invention is intended to overcome these problems, and to provide further advantages which a person of ordinary skill in this art will appreciate upon reading this disclosure.

More specifically, in one preferred embodiment, a liquid dispensing system is disclosed which dispenses the “first draw” liquid at an appropriate temperature. In a preferred embodiment, after the user requests dispensing, such as by depressing or actuating a dispensing button or lever, a “pause” or delay may be provided before liquid is dispensed from the (e.g., cold or hot) faucet(s) or dispenser(s); during this dispensing pause or delay, the liquid may be moved from a residual collection point (such as in the tubing between a storage tank and the faucet, which may have been sitting in the tubing for some time, and may not be at the optimum target temperature). After periodical flushing of the residual water from its collection point(s) in the waterways, the appropriate waterway(s) may be replaced with “fresh” liquid at the target temperature drawn from a separate source (such as a cold, hot or room temperature tank or other tank or source), in order to eliminate the growth in the system waterways of bacteria, algae or any other organic material or pathogen in between the times of customer dispensing, that may otherwise cause taste and/or odor problems and/or health issues to the user.

In a particularly preferred embodiment of the invention, a pump-fed, liquid dispensing system is provided with at least one liquid storage tank (e.g., a cold tank containing a chilled liquid such as water). A first liquid communication means, such as plastic or stainless steel tubing, is provided to allow the liquid to flow along a first path between the liquid storage tank and a dispenser. A first valve may be located along this first path. A second liquid communication means allows the liquid to flow from the first valve to the storage tank. One or more pumps may be used to selectively draw the liquid from the storage tank and cause it to flow along the first path, as controlled by the user. When liquid from the storage tank is selected by the user to be dispensed, a dispensing delay may be caused to occur after the one or more pumps is actuated and before the corresponding valve is opened. This delay enables residual liquid in the corresponding liquid communication means to flow into the storage tank before the liquid is dispensed, to thereby provide a dispensed liquid having a temperature which is substantially the same as the temperature of the liquid in the storage tank.

The foregoing system may also be provided with multiple tanks (e.g., cold and hot tanks), and a second liquid communication means allowing the liquid to flow along a second path between the second (e.g.) hot tank and either the same or a different dispenser. A second valve may be located along the second path. A third liquid communication means may be provided to allow the liquid to flow from the second valve to the first (e.g., cold) tank. The one or more pumps (such as but not limited to a dedicated cold water pump and a dedicated hot water pump) may be used to selectively draw the liquid from the hot tank and to cause it to flow along the second path, as controlled by the user. When liquid from the cold or hot tank is selected by a user, a delay occurs after the one or more pumps is actuated and before the corresponding valve is opened, enabling residual liquid in the corresponding liquid communication means to flow into the selected cold or hot tank before the liquid is dispensed, to thereby provide a dispensed liquid having a temperature which is substantially the same as the temperature of the liquid in the desired tank. The principles of this embodiment of the present invention may also be employed when water is dispensed from a pressurized system such as a municipal water supply, such that residual water in the tubing is periodically flushed and replaced with “fresh” water during non-dispensing periods.

Various exemplary embodiments may be used with the foregoing systems, which also may be modified as discussed below. For example, each of the first, second, third and fourth liquid communication means may include plastic tubing. As another example, the cracking pressure of the first and second valves may be in a predetermined range, such as about 2 ounce-force, depending on other variables such as water pump pressure and dispensing flow rate. The dispensing delay may vary, but one useful range is between about 0.5 and 1.5 seconds, such as about 1.2 seconds.

In another embodiment of the present invention, a gravity-fed liquid dispensing system is provided. The system may include at least one cold tank containing a chilled liquid, a first liquid communication means allowing the liquid to flow under the influence of gravity, as actuated by a user, along a first path between the at least one cold tank and a dispenser, a first valve located along the first path, and a second liquid communication means allowing the liquid to flow from the first valve to the cold tank. When liquid from the cold tank is selected by a user, a predetermined delay may occur before the corresponding valve is opened, enabling residual liquid in the corresponding liquid communication means to flow into the cold tank before the liquid is dispensed, to thereby provide a dispensed liquid having a temperature which is substantially the same as the temperature of the liquid in the desired tank. The gravity-fed system may source water from an inverted water bottle in fluid communication with the cold tank, for example.

The gravity-fed, liquid dispensing system may also include a hot tank containing heated liquid, a third liquid communication means allowing the liquid to flow along a second path, as actuated by a user, between the hot tank and either the same or a different dispenser, a second valve located along the second path, and a fourth liquid communication means allowing the liquid to flow from the second valve to the cold tank. When liquid from the cold or hot tank is selected by the user, a predetermined delay may be caused to occur before the corresponding valve is opened, enabling residual liquid in the corresponding liquid communication means to flow into the selected cold or hot tank before the liquid is dispensed, to thereby provide a dispensed liquid having a temperature which is substantially the same as the temperature of the liquid in the desired tank.

### Stay-Cool™

In yet another embodiment of the present invention, a liquid dispensing system capable of dispensing chilled liquid is provided with the ability to maintain the liquid in the cold waterway at a desired, optimum target temperature (such as a temperature to minimize or eliminate pathogen growth). In a preferred embodiment, this may be accomplished by moving liquid out of the cold waterways, such as by running a pump for about 30 seconds every 30 minutes, so that water at a temperature above the target temperature is removed from the cold waterways and replace with chilled water at or below the target temperature. The replacement water may be taken from a reservoir or storage tank, such as a cold tank, for example.

In a preferred embodiment, a liquid dispensing system includes at least one cold tank, a first liquid communication means allowing the liquid to flow along a first path between the cold tank and a dispenser, a valve located along the path, and a pump for drawing the liquid from the cold tank and causing it to flow along the first path as controlled by the user. In this embodiment, the pump automatically activates on a periodic basis to flush the liquid in the first liquid communication means and replace it with cold tank (or externally-sourced and chilled) liquid keep the temperature of the liquid within the first liquid communication means below a certain predetermined temperature designed to limit the growth of microorganisms within the liquid. As a non-limiting example, the pump may automatically activate for less than about five percent of a total operating time of the system, such as about 5 seconds during each consecutive 30 minute period to accomplish this result.

Liquid dispensing systems capable of operating in the above-described Accu-Temp™ and/or Stay-Cool™ modes may also be used to move either hot liquid or ozonated liquid through the waterways, as further explained below.

### Ozone Sanitizing

In still another embodiment of the present invention, a method is provided for sanitizing a liquid dispensing system using ozonation. In general, ozone may be used to sanitize the entire waterway system of the liquid dispensing system, including the nozzle/dispensing mechanism. Ozone sanitization may be employed with systems including a cold tank only, systems employing cold, hot (and/or other tanks), or systems involving water supplied from pressurized municipal supply systems.

In an exemplary embodiment of a liquid dispensing system using at least one cold tank and at least one hot tank, a first liquid communication means may also be provided, allowing the liquid to flow along a first path between the cold tank and a dispenser. A second liquid communication means may also be provided, allowing the liquid to flow along a second path between the hot tank and either the same or a different dispenser. A first valve may be located along the first path, and a second valve may be located along the second path. A third liquid communication means may also be provided, allowing the liquid to flow from the first valve to the cold tank, and a fourth liquid communication means may be used to allow the liquid to flow from the second valve to the cold tank. One or more pumps may be used to selectively draw the liquid from each of the tanks and cause it to flow along the first path or the second path, as controlled by the user. The liquid-contacting portions of the system may be sanitized by ozonating the liquid, using any convenient and available ozonation system. The liquid within the cold tank may also directly ozonated, and then this ozonated liquid may be used to sanitize the remaining liquid and liquid-contacting parts within the system.

### BRIEF DESCRIPTION OF THE DRAWINGS

The novel features which are characteristic of the invention are set forth in the appended claims. The invention itself, however, together with further objects and attendant advantages thereof, can be better understood by reference to the following description taken in connection with the accompanying drawings, in which:

FIG. 1 is a schematic diagram of one preferred embodiment of the present invention;

FIG. 2 is a schematic diagram of a preferred embodiment of the present invention which is an alternative to that of FIG. 1, and that involves what is termed here SIP/Ozonated water sanitization;

FIG. 2A is an exploded view of the corresponding circled portion shown in FIG. 2;

FIG. 3 is a perspective view of exemplary union-T fitting which may be used with the present invention;

FIG. 4 is a sectional view of an exemplary spring-loaded check valve which may be used with the present invention to provide a suitable cracking pressure for the valve;

FIG. 5 is a schematic view showing another preferred embodiment of a liquid dispensing system of the present invention (described here as Accu-Temp™); and

FIG. 6 is a schematic view showing another preferred embodiment of a liquid dispensing system of the present invention, running in other preferred modes (described here as Stay-Cool™ and SIP sanitization).

The components in the drawings are not necessarily to scale, emphasis instead being placed upon clearly illustrating the principles of the present invention. In the drawings, like reference numerals designate corresponding parts throughout the several views.

### DETAILED DESCRIPTION OF THE PREFERRED EMBODIMENTS

Set forth below is a description of what are believed to be the preferred embodiments and/or best examples of the invention claimed. Future and present alternatives and modifications to this preferred embodiment are contemplated. Any alternatives or modifications which make insubstantial changes in function, in purpose, in structure, or in result are intended to be covered by the claims of this patent.

The water cooler systems or liquid dispensing systems of the present invention may utilize any commercially available water coolers or liquid dispensing systems, including those disclosed in the above-referenced patent applications which are incorporated by reference into this application. The specific embodiments discussed below are not intended to narrow the scope of the claimed invention as discussed in the Summary of the Invention, above.

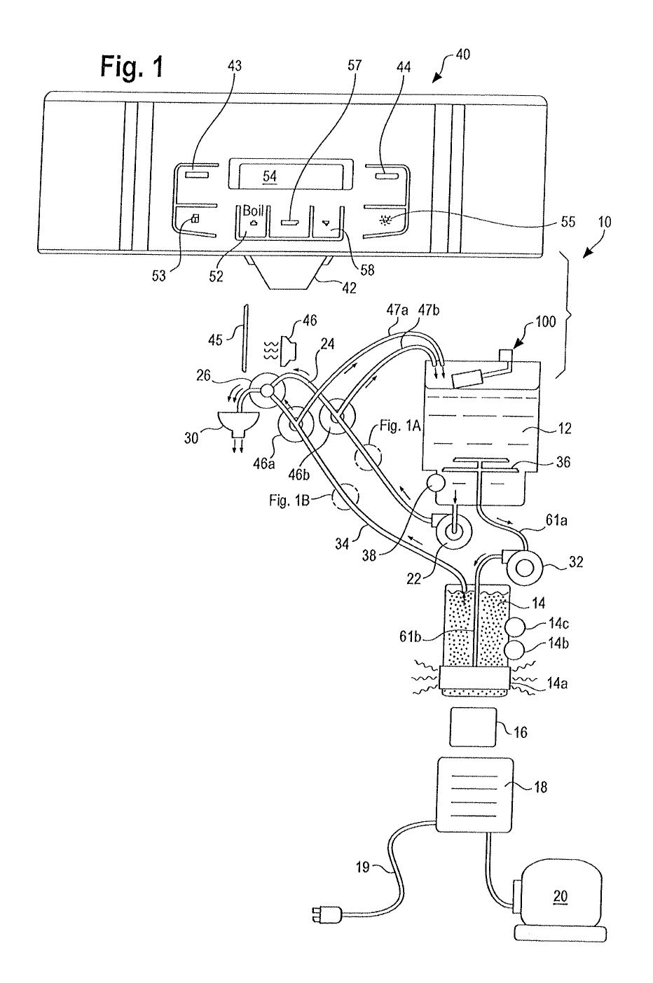

Referring first to FIG. 1, one preferred embodiment of the water cooler system of the present invention, generally referred to by reference numeral 10, is shown. Water cooler system 10 may generally include cold tank 12, hot tank 14, float system 100 for detecting a water level in tank 12, transformer or power adapter/switching power supply 16, control PCB 18, and compressor 20. (A float system may be used for a point-of-use (POU) system supplied by pressurized water instead of a bottled water. The float system can be replaced/exchanged with the alternate system disclosed here for the bottle water version application, with the rest of the system remaining the same. For example, if a “bottom load” system is used in which the water bottle is located below the dispenser, a pump may be used to move the water to the cold tank, and a float system may be used to regulate when to turn on/turn off the pump based on the sensed level in the cold tank.) In an alternative embodiment, a water regulating component commercially known as “EZ-Fill” may be used in connection with inverted bottle dispensing.

By energizing cold water pump 22 and cold-side, 3-way solenoid valve 46b, cold water may be pumped from the cold tank through cold water dispensing tubing 24, and then may be dispensed using 3-way solenoid 26, in a manner well known in the art, at spout 30. Alternatively, by energizing hot water pump 32 and hot-side, 3-way solenoid valve 46a (which may also be a mechanical valve), hot water may be pumped from the hot tank through hot water dispensing tubing 34, and then dispensed in a similar manner to that of the cold water.

Cold tank 12 may include a baffle 36, which may but need not be the type of baffle disclosed in copending U.S. Ser. No. 12/555,866. Cold tank 12 may also include cold control sensor 38 which may be used to turn on/off compressor 20 in order to maintain the cold tank water at the desired temperature. Hot tank 14 may include a heater band 14a, a heat limiter 14b (e.g., a thermostat to cut off power if the hot tank temperature exceeds a predetermined temperature, such as 95° C./203° F.), and a NTC thermistor 14c, such as a negative temperature coefficient thermistor). (A thermocouple may be used here instead, to provide a similar function.) NTC thermistor 14c may be used to sense the temperature of the hot tank, electronically communicating with control PCB 18 to control the hot tank temperature to be (e.g.) 60° C./140° F. for energy-saving mode, 86° C./187° F. for normal hot water cycle, and 92° C./198° F. for Insta-Boil purposes, described below).

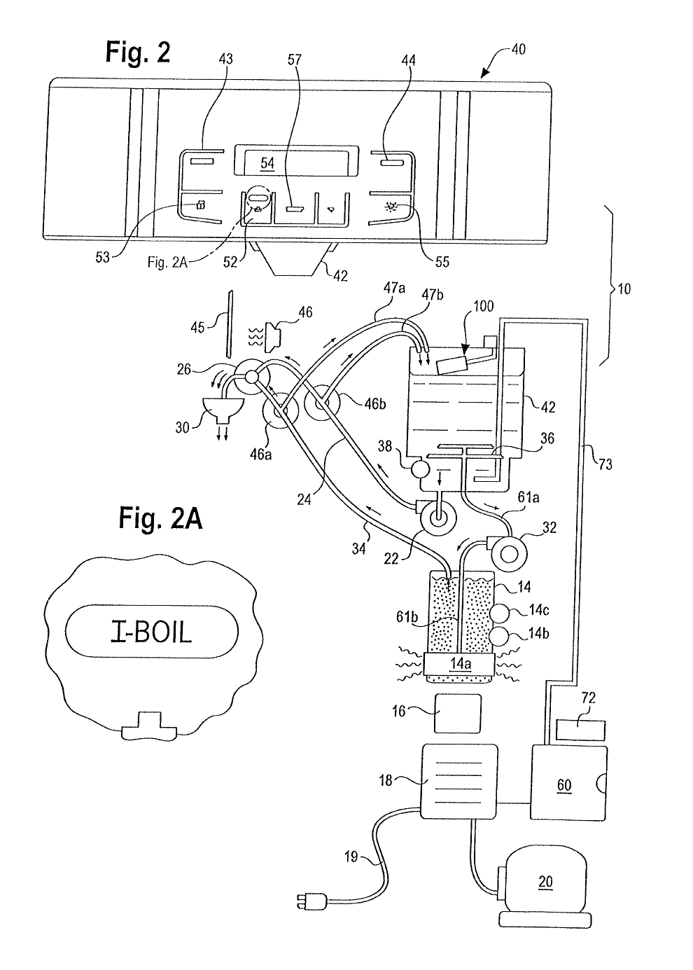

Referring to FIG. 2, control PCB 18 may include a SIP black box 60 and SIP air pump 72. In the exemplary embodiment, control PCB 18 operates as the brain of the unit to organize the components (heat band, compressor, pump, solenoid, LED, LCD, SIP, etc) and provide the features the user needs. SIP box 60 may be an ozone generator, which generates ozone to sanitize the system, such as through ozone tubing 73 (FIG. 2).

Referring to FIGS. 1-2, water cooler 10 may include a graphical (e.g., LED) interface 40 such as shown, which may include: a safety lock indicator 42; hot (43) and cold (44) water indicators which may remain always on; an interface PCB 45 which may be used to mount all the tactile switches, LEDs and LED, allowing the user to interface with the unit; a buzzer 46 for providing audible tones (which may have a decibel adjustment feature); and an i-Boil button 52.

### Accu-Temp™

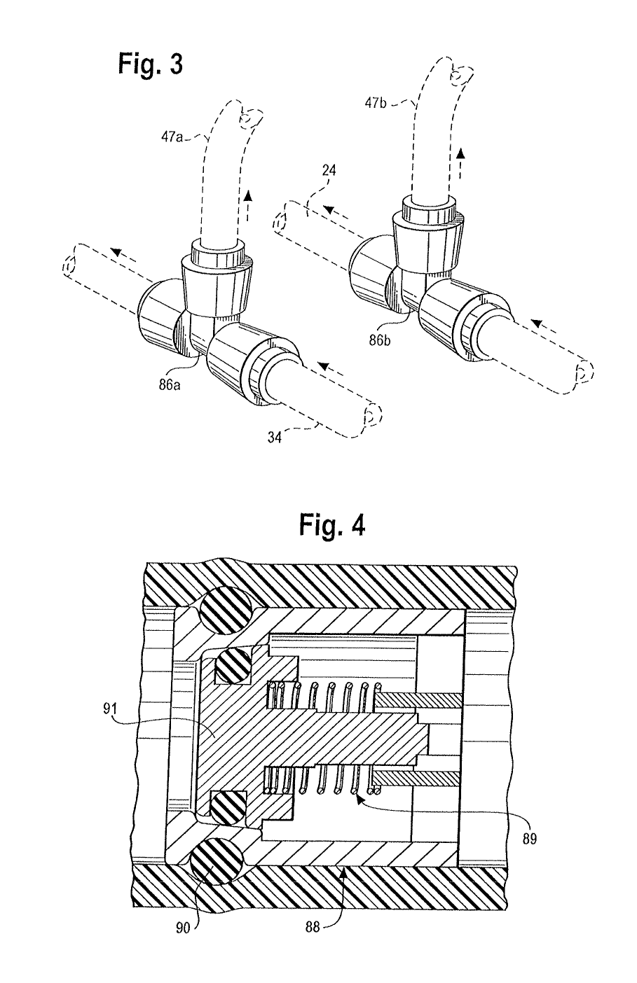

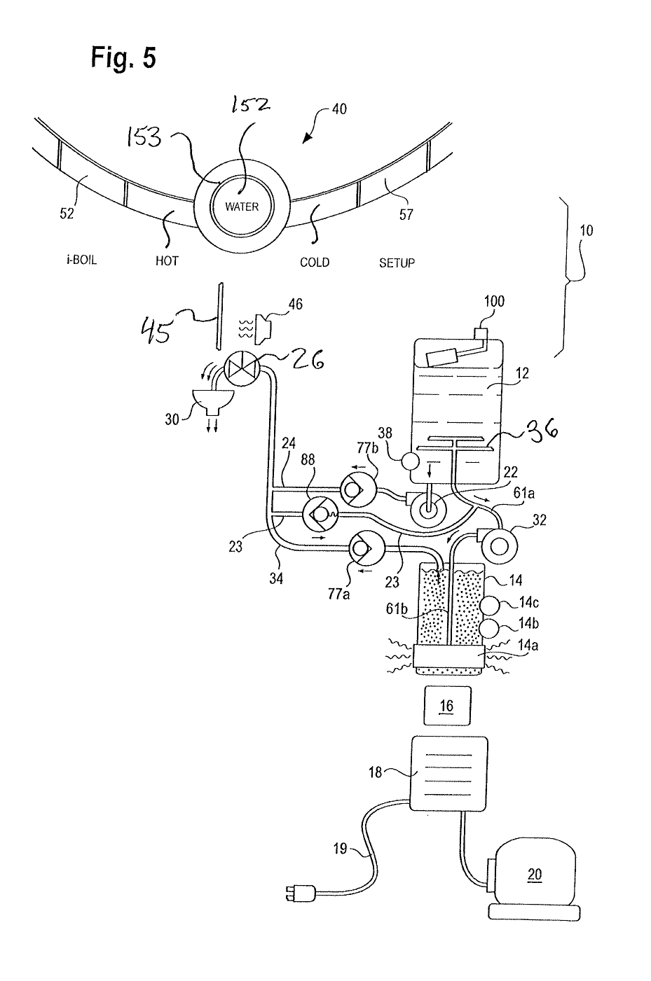

Referring now to FIGS. 1-2 and 5, one preferred embodiment of water cooler system 10, termed here the “Accu-Temp™” feature, is designed to ensure that the dispensed water exiting the dispensing spout has a temperature very close to that of the temperature in either the cold tank (for cold water) or the hot tank (for hot water). For this purpose, water pumps 22, 32 and dispensing solenoid valve 26, already present in the pump-fed system, may be used. In addition, solenoid valves 46a, 46b (FIGS. 1-2) may be replaced with union-T fitting 86a, 86b (FIG. 3), and spring-loaded, flow-restricting, check valve assemblies 88 (FIGS. 4 and 5) may be used to achieve a low cracking pressure. If used in the embodiments shown in FIGS. 1-2, the check valves may be located inside return tubing 47a and 47b, and may be small, cylindrically-shaped components as shown. (Instead of check valves, solenoid valves may be used.)

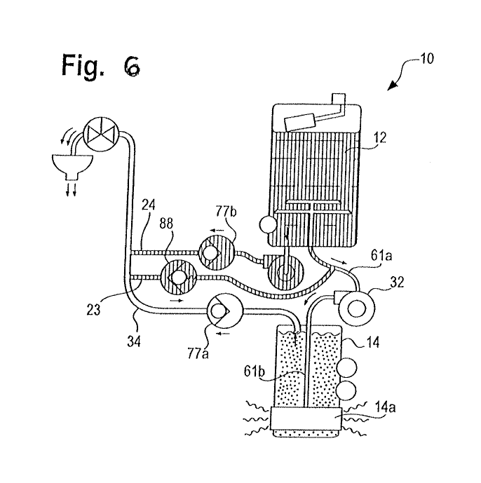

FIG. 4 is one example of a spring-loaded check valve 88 which may be used, and which includes spring 89, O-ring 90 and plunger 91. Referring to FIGS. 5 and 6, flow-restricting check valves 77a, 77b may be used. Check valve 88 preferably has a low-cracking pressure, which may be, as a non-limiting example, in the 2-3 ounce-force range (most preferably 2 ounce-force, for example, and which may vary depending on the water pump pressure and dispensing rate).

Referring to FIG. 3, return tubing 47a, 47b may be provided to create a loop/path for the residual room temperature water in the tubing to flow back to the cold tank. When water pump (22 or 32) is on but dispensing solenoid valve 26 is closed/off, water may flow through return tubing (47a or 47b) and return back to the cold tank. When dispensing solenoid valve 26 is on/open and water pump 22 or 32 is on, the water may flow through dispensing tubing (24 or 34) to dispensing spout 30, not through return tubing 47a or 47b. Each time a user depresses a button or switch for dispensing water, the water pump may first actuate to begin pumping water, but the dispensing solenoid valve may not open immediately. Instead, there may be a lag time before the dispensing valve is actuated (e.g., of about 0.5-1.5 seconds, preferably about 1 second, with the lag time based on tubing length and insulation, as persons of ordinary skill in the art can readily calculate). During this lag time, the residual room temperature water in the tubing may flow through the newly-added, solenoid valve (46a or 46b) or low-cracking pressure check valve 88 and return tubing (47a or 47b in the FIGS. 1-2 embodiment, and 23 in the FIG. 5 embodiment) and return to the cold tank. After this lag time has elapsed, the dispensing valve may then open and the water may then be dispensed. At this moment, the water dispensed from the spout may have very little mix with the residual room water and may have a water temperature very close to that of the water in the corresponding, desired (cold or hot) tank.

Referring now to FIG. 5, water cooler 10 may include a interface 40 such as shown, which may include: a water dispensing button 152; a LED ring indicator 153 for hot and cold which will only be lit when the Hot (red ring) or Cold (blue ring) water function is first selected, prior to depressing water dispensing button 152; an interface PCB 45 which may be used to mount all the tactile switches and LEDs, allowing the user to interface with the unit; a buzzer 46 for providing audible tones (which may have a decibel adjustment feature); and a boiling water button 52. The arrows on check valves 77a, 77b and 88 indicate flow direction. Cold pump 22 supplies liquid to cold tank 12 (which includes buffer 36 and float valve 100), while hot pump 32 supplies liquid to hot tank 14. Solenoid valve 26 coordinates dispensing of liquid through dispenser 30.

In the prototype of the drawings described here, testing showed that the water temperature between dispensing spout 30 and the water tanks (12 or 14) could be controlled within a range of +/−2° C./3.6° F.

Again, as described above, one preferred embodiment may dispense pressurized liquid from a municipal supply, for example, without using local storage tanks, and simply periodically flush residual liquid from the dispensing tubing, and replace this flushed liquid with “fresh” liquid which may be chilled or heated to avoid pathogen build-up. If a localized dispensing system is employed utilizing one or more storage tanks, such as a cold tank, a cold and hot tank, or other tanks, the flushing liquid may be drawn from one of these tanks, or it may be drawn from an external source.

Those of ordinary skill in the art will also understand that the predetermined delay necessary to allow the flushing and/or liquid replacement steps to occur (with regard to the residual liquid that has collected in the waterways in between dispensing times) can be varied to permit substantially all, but not necessarily all, of the residual liquid to be removed from the waterways.

### Stay-Cool™

Referring to FIGS. 5 and 6, in another embodiment of the present invention, which may be used alone or in conjunction with the above-referenced “Accu-Temp™” feature, termed here the “Stay-Cool™” system, water cooler system 10 may be designed to ensure that the water in the cold tubing and waterway areas may be maintained below a certain predetermined temperature chosen to limit or prevent bacteria growth. As mentioned above, water sitting in the tubing/waterway areas, when substantial time periods go by in between dispensing, can drop to room temperature warm or warmer (as the temperature inside a unit is warmer, due to the heat from the hot tank and the compressor), and that warm temperature environment can be a good breeding ground for bacteria and algae growth. For example, if 3-6 months go by in between sanitization of these areas, it is likely that these areas contain substantial amounts of bacteria and/or algae already.

In one embodiment of the “Stay-Cool™” system, the water pump may periodically be activated for a predetermined time period (e.g., for 5 seconds every 30 minutes, with the actual timing to be fine-tuned based on tubing length and insulation, as will be understood by those designers of ordinary skill in the art) in order to keep the water in the cold waterways cool and to suppress bacteria and algae growth. Referring to the embodiment shown in FIGS. 5 and 6, return tubing 23 may be used to provide a path for the warm, residual water in the tubing to periodically flow back to the cold tank as the warm, residual water in the tubing is periodically flushed with cool water from the cold tank. This feature can be used alone or be designed to work with the Accu-Temp™ feature to reduce the lag/waiting time for the Accu-Temp™ feature. This feature need not be used in the hot waterways, as hot water will kill bacteria and algae every time it runs through the hot waterways.

### Ozone Sanitization

In yet another preferred embodiment of the present invention, a water cooler system may be provided which sanitizes the cold waterway system and wetted parts by using ozonated water generated by an ozone generator. As examples, SIP 3207 from SIP Technologies, or another ozone sanitization unit, may be used. (Sanitization of the hot waterway system and wetted parts is not needed as the hot water will prevent the growth of bacteria and algae.) Using the waterway tubing and components shown in FIG. 1, for example, activating cold water pump 22 only (not dispensing solenoid valve 26) will redirect the cold water back to the cold tank. When the cold water is ozonated within the cold tank, circulating the ozonated water throughout the cold system will sanitize the entire cold waterway (including cold waterways, cold Accu-Temp™ tubing 47b, the cold pump and the cold solenoid valve 46b) and the wetted parts that touch ozonated water. During the ozone sanitization mode, the system may also run hot water sanitization on the hot side, so that hot water will run through the hot water tubing to sanitize the hot water waterway portion, allowing both cold and hot side of the waterways to be sanitized.

In an exemplary embodiment, when the unit is placed in SIP Manual Clean mode, the user places a vessel capable of holding (e.g.) 4 cups/1 liter of water under the nozzle area. When the SIP Manual Full Clean mode is activated (such as by depressing the “Manual Full Clean” white button on the back of the unit for about 3 seconds and waiting for an audible tone), “Full Clean On” will be displayed, and the entire waterways will be sanitized. During Auto Clean mode, there is a cold waterway section that is not sanitized. Also, the Auto Clean mode does not require user intervention (sanitization will automatically begin based on the Frequency and Start time setup), while the Manual Clean mode does.

When the SIP sanitization feature is activated (either SIP Auto Clean or SIP Manual Full Clean modes), the compressor may stop, and the “Auto Clean On” or “Full Clean On” terminology may be displayed. At both SIP Manual and SIP Auto Clean modes, the system may wait about 30 minutes for any ice bank in the cold tank to dissolve before providing power to the SIP black box (e.g., SIP model 3207). The SIP black box may be provided power through power cord for 75 minutes. The SIP black box may finish the cycle and stop automatically roughly after 75 minutes. When the SIP black box is powered (in both SIP Manual and SIP Auto Clean modes), the compressor may resume its work to cool down the water temperature and facilitate ozone absorption. About 40 minutes later (or when the ozone concentration in the water has reached a certain desired level), the cold water pump may commence running for 2 seconds every 10 seconds for 20 minutes. Ozonated cold water will be circulated back to the cold tank through the cold Accu-Temp™ tubing. At the 55-minute mark, the i-Boil feature may be activated once, in order to raise the hot tank temperature to (e.g.) 92° C./198° F., and sanitize the hot tank. At the 60-minute mark, the hot water pump may run for 1 second, and then stop 2 seconds, and repeat this cycle 15 times. Ozonated water may flow through the baffle waterway to the hot tank, to sanitize the baffle waterway. Hot water may exit the hot tank, to also sanitize the hot Accu-Temp™ tubing. 75 minutes after the SIP black box is activated, the system may return to its normal operational mode.

During the SIP Auto Clean and SIP Manual Clean sanitization processes, both hot and cold dispensing buttons will be locked out, and all LEDs will be powered off, except the LCD display and backlight. Either “Auto Clean On” or “Full Clean On” will be displayed, depending on which was selected. Attempts to dispense water may trigger a buzzer. The setup buttons may be used. Either mode may be stopped/exited by depressing and holding the “Manual Full Clean” white button on the rear of the unit for (e.g.) 3 seconds until a tone is heard.

During the SIP Manual Clean sanitization process, in an exemplary embodiment, at the 58-minute mark, the cold solenoid may be opened and the cold pump energized to dispense ozonated water from the nozzle for 1 second, and then stop dispensing for 2 seconds, with this cycle repeated 15 times, allowing the ozonated water to flow through the nozzle and sanitize this area. At the 59-minute mark, the hot solenoid may be opened and the hot pump energized to dispense hot water from the nozzle for 1 second, and then to stop dispensing for 2 seconds, with this cycle repeated 15 times, allowing the ozonated water to flow through the baffle waterway to the hot tank, to sanitize the baffle waterway, and to allow the hot water to flow through the nozzle and sanitize this area.

Any ozone-sanitizing liquid dispensing system according to the present invention may be used to sanitize the faucet/nozzle/dispensing mechanism, as desired.

Incorporating by reference the applicable disclosure in pending U.S. Ser. No. 12/116,407, filed May 7, 2008, titled “Bottom Load Water Cooler,” and referring back to graphical interface 40 in FIGS. 2, 2A and 5, water cooler 10 may be designed so that pressing and holding “Insta-Boil” or “i-Boil” button 52 (e.g., depressing for one second may result in an audible tone or buzzer) results in the “i-Boil” mode being activated (dispensing button 43 with red LED light may also blink, for example), causing the water in the hot tank to be heated (e.g., to 92° C./198° F.); the user may be signaled when the water is sufficiently heated (e.g., the red LED may return to steady and/or a long beep may sound), such as for tea, instant noodles, and other applications requiring hotter water.

As to filter change, when one of the dispensing buttons 43, 44 is pressed and held, for example, a message may appear in graphical display area 54 (e.g., “Premium Quality Water” or “Filter OK”) indicating that the filter life is still acceptable; “Schedule Service Call” or similar terminology may appear if the filter needs to be replaced.

Referring to FIG. 2, in the exemplary embodiment, interface 40 may be provided with a button 53 with the “lock” icon, such that by pressing and holding this button, for example (and, e.g., waiting for an audible tone) can lock or unlock hot water dispensing, as desired. A brightness button 55 may also be provided, allowing the user to adjust the brightness of all LEDs and LCD displays (e.g., there may be three brightness modes: full, 50% and 10%).

Still referring to FIG. 2 and interface 40, depressing “setup” button 57 and (e.g.) waiting for an audible tone, can activate the setup mode. Now, by using up button 52 (which may be integrated with the “i-Boil” button) and down button 58, the desired settings (e.g., time, date, start, stop and frequency times for various functions such as sanitizing, auto clean, etc.) may be input. An “energy saved” switch may also be provided, in order to lower the hot tank temperature (e.g., to 60° C./140° F.) during the night to save additional energy.

As referenced above, when the SIP sanitization feature is actuated (either in the Auto Clean or Manual Clean modes), in the exemplary embodiment, the compressor may stop running, and the “SIP Auto Clean ON” or “SIP Manual Clean ON” terminology may appear in graphical display 54 on interface 40. After waiting for about 30 minutes for any ice bank to dissolve, SIP black box 60 (FIG. 2) may be provided with power using power cord 19 from control PCB 18, for 75 minutes. 40 minutes later, cold water pump 32 may be energized to run for 2 seconds every 10 seconds, for a 20-minute period. Ozonated cold water may now be circulated back to cold tank 12 through the cold Accu-Temp™ tubing 47b. 75 minutes after SIP black box 60 is activated, system 10 may return to its normal operational mode.

Again, during the SIP Auto Clean sanitization process, all dispensing buttons may be locked out, and all LEDs may be turned off, except for the LCD display with the “SIP Auto Clean ON” terminology. Attempts to dispense will not work, and may trigger an audible response (e.g., a buzzer with 3 quick beeps in ½ seconds). Again, the setup buttons will be useable.

In contrast, during the SIP Manual Clean sanitization process, some water will be dispensed from nozzle, all LEDs may be powered off except the LCD display and backlight, and “Full Clean On” may show in the LCD display. Attempts to dispense water may trigger a buzzer and/or audible tone. Other setup features (setup, up, down, brightness) may still be used. In the exemplary embodiment, at the 58-minute mark, the cold solenoid may be energized and the cold pump caused to dispense ozonated water from the nozzle for 1 second, and then stop dispensing for 2 seconds, and this cycle may be repeated 15 times, allowing the ozonated water to flow through the nozzle and sanitize this area. At the 59-minute mark, the hot solenoid may be opened/energized and the hot pump caused to dispense hot water from the nozzle for 1 second, to stop dispensing for 2 seconds, and to repeat this cycle 15 times, causing the hot water to flow through the nozzle and sanitize this area.

In an alternative embodiment, not shown in the drawings, the hot tank may be eliminated, such that the water cooler only provides room temperature and chilled water. In this embodiment, pump 32 may communicate directly with cold water tubing 34.

The above description is not intended to limit the meaning of the words used in the following claims that define the invention. Other systems, methods, features, and advantages of the present invention will be, or will become, apparent to one having ordinary skill in the art upon examination of the foregoing drawings, written description and claims, and persons of ordinary skill in the art will understand that a variety of other designs still falling within the scope of the following claims may be envisioned and used. It is contemplated that these or other future modifications in structure, function or result will exist that are not substantial changes and that all such insubstantial changes in what is claimed are intended to be covered by the claims.

The following terms are used in the claims of the patent as filed and are intended to have their broadest meaning consistent with the requirements of law. Where alternative meanings are possible, the broadest meaning is intended. All words used in the claims are intended to be used in the normal, customary usage of grammar and the English language.

We claim:

1. A liquid dispensing device capable of being used to perform the following steps:
dispensing a liquid through a dispensing mechanism that is in fluid communication with a first liquid communication means; and
periodically, automatically flushing liquid collecting in the first liquid communication means from the first liquid communication means during a time when no dispensing of the liquid has occurred, to facilitate the prevention of pathogen contamination within the liquid contained in the first liquid communication means during non-dispensing time periods.

2. The liquid dispensing device of claim 1, wherein the dispensing device comprises either a pump-fed or gravity-fed liquid dispensing system.

3. The liquid dispensing device of claim 1, wherein the device includes at least one cold tank containing a chilled liquid, and the first liquid communication means allows the liquid to flow along a first path between the at least one cold tank and the dispensing mechanism.

4. The liquid dispensing device of claim 3, wherein the device also includes at least one hot tank contained a heated liquid, and a second liquid communication means allows the liquid to flow along a second path between the at least one hot tank and the dispensing mechanism, and wherein the periodic flushing step may include the step of flushing liquid collecting in the second liquid communication means from the second liquid communication means during a time when no dispensing of the liquid has occurred, to facilitate the prevention of pathogen contamination within the liquid contained in the second liquid communication means during non-dispensing time periods.

5. The liquid dispensing device of claim 1, further comprising the step of causing a delay in dispensing to occur prior to dispensing and after the request for dispensing has been made, to provide the device with time to perform the flushing step.

6. A liquid dispensing device capable of dispensing a liquid through a dispensing mechanism that is in fluid communication with a first liquid communication means, and also capable of being used to perform the following steps:
prior to dispensing the liquid, and after a request for dispensing has been made to the system by an operator of the system, automatically flushing liquid that has collected in the first liquid communication means from the first liquid communication means; and
replacing the flushed liquid in the first liquid communication means with liquid at a predetermined, desired temperature.

7. The liquid dispensing device of claim 6, wherein the dispensing device comprises either a pump-fed or a gravity-fed liquid dispensing system.

8. The liquid dispensing device of claim 6, wherein the device includes at least one cold tank containing a chilled liquid, the first liquid communication means allows the liquid to flow along a first path between the at least one cold tank and the dispensing mechanism, and the replacement liquid is at a temperature which is substantially the same as the temperature of the liquid in the cold tank.

9. The liquid dispensing device of claim 8, wherein the device also includes at least one hot tank contained a heated liquid, and a second liquid communication means allows the liquid to flow along a second path between the at least one hot tank and the dispensing mechanism, and wherein the automatic flushing step may include the step of flushing liquid collecting in the second liquid communication means from the second liquid communication means, and wherein the replacing step may include the step of replacing the flushed liquid from the second liquid communication means with liquid at a predetermined, desired, heated temperature which is substantially the same as the temperature of the liquid in the hot tank.

10. The liquid dispensing device of claim 6, further comprising the step of causing a delay in dispensing to occur prior to dispensing and after the request for dispensing has been made, to provide the device with time to perform the flushing and replacing steps.

11. The liquid dispensing device of claim 10, further comprising one or more valves in fluid communication with the first liquid communication means, wherein the one or more valves participate in causing the dispensing delay.

12. The liquid dispensing device of claim 11, wherein at least one of the one or more valves has a cracking pressure in the range of about 2-3 ounce-force.

13. The liquid dispensing device of claim 11, wherein the device includes one or more pumps, and wherein the step of causing a delay in dispensing to occur includes the step of actuating the one or more pumps before opening the one or more valves, enabling residual liquid in the first liquid communication means to flow out of the first liquid communication means.

14. The liquid dispensing device of claim 9, wherein the first and second liquid communication means comprise plastic or stainless steel tubing.

15. The system of claim 10, wherein the dispensing delay is in the range of about 0.5-1.5 seconds.

16. A liquid dispensing system, comprising:
a liquid supply;
a first liquid communication means allowing the liquid to flow along a first path from the liquid supply to a dispenser;
a pump for drawing the liquid from the liquid supply and causing it to flow along the first path as controlled by the user, the pump automatically activating on a periodic basis during a non-liquid-dispensing mode to flush liquid in the first liquid communication means from the first liquid communication means, and to replace the flushed liquid with chilled liquid, in order to maintain a temperature of the liquid within the first liquid communication means below a certain predetermined temperature to limit the growth of pathogens within the liquid.

17. The liquid dispensing system of claim 16, further comprising a second liquid communication means communicating with the first liquid communication means and providing a path permitting residual, warmer liquid in the first communication means to be returned to a cold tank.

18. The liquid dispensing system of claim 16, wherein the pump automatically activates during the non-liquid-dispensing mode for a period of time which is less than about five percent of a total operating time of the system.

19. A method for sanitizing a liquid dispensing system, comprising the steps of:
providing a liquid supply source;
providing a first liquid communication means allowing the liquid to flow along a first path between the liquid supply source and a dispenser; and
sanitizing substantially all of the waterways of the system and the dispenser by sufficiently ozonating the liquid within the waterways.

20. The method of claim 19, wherein the liquid supply source comprises at least a cold tank, and wherein the liquid within the cold tank is ozonated, and then this ozonated liquid is used to sanitize the remaining liquid and liquid-contacting parts within the system.
